(19) United States
(12) Patent Application Publication
(10) Pub. No.: US 2026/0331224 A1
(43) Pub. Date: Oct. 22, 2026

(54) MACHINE LEARNING IDENTIFICATION OF MUTATIONAL SIGNATURES

(71) Applicant: HADASIT MEDICAL RESEARCH SERVICES AND DEVELOPMENT LTD., Jerusalem (IL)

(72) Inventors: Adar YAACOV, Jerusalem (IL); Shai ROSENBERG, Jerusalem (IL)

(21) Appl. No.: 19/112,085

(22) PCT Filed: Sep. 14, 2023
(86) PCT No.: PCT/IL2023/051009

§ 371 Date: Mar. 14, 2025

Related U.S. Application Data
(60) Provisional application No. 63/406,909, filed on Sep. 15, 2022.

(51) Int. Cl. G16B 40/20; G16B 20/50; G16H 20/00; G16H 50/30

(52) U.S. Cl. CPC G16B 40/20; G16B 20/50; G16H 20/00; G16H 50/30

(57) ABSTRACT

A computer-implemented method comprising: receiving, as input, a plurality of DNA sequencing samples corresponding to a cohort of subjects having various tumor types; annotating each of the DNA sequencing samples with annotations indicating (a) a tumor type associated with the DNA sequencing sample, (b) one or more mutations represented in the DNA sequencing sample, and (c) at least one dominant mutational signature associated with the DNA sequencing sample; at a training stage, training a machine learning model on a training dataset comprising: (i) the DNA sequencing samples, and (ii) labels indicating the annotations; and at an inference stage, applying the trained machine learning model to a target DNA sequencing sample from a target subject, to predict a dominant mutational signature associated with the target DNA sequencing sample.

### CROSS-REFERENCE TO RELATED APPLICATIONS

This application claims priority from U.S. Application Ser. No. 63/406,909, filed Sep. 15, 2022, entitled “MACHINE LEARNING IDENTIFICATION OF MUTATIONAL SIGNATURES,” the contents of which are hereby incorporated herein in their entirety by reference.

### FIELD OF THE INVENTION

The present invention relates to the field of machine learning.

### BACKGROUND

Somatic mutations accumulate through multiple mutational processes, creating patterns termed ‘mutational signatures.’ In cancer, mutational signatures reflect underlying processes. Some somatic mutations, such as SBS1 and SBS5, are endogenous, ubiquitous, and age-correlated. Other mutations reflect a unique exogenous or endogenous process, such as the UV-related signatures, APOBEC-related signatures, and others.

Mutational signatures can provide significant prognostic and therapeutic insights. For example, In a clinical use-case, APOBEC signature was identified as a robust and specific predictor for resistance to EGFR-TKIs. Similarly, patients with APOBEC or homologous recombination deficiency signatures may benefit from ATR inhibitors or PARP inhibitors. In another example, immune checkpoint inhibition may be beneficial for tumors harboring UV, Tobacco, APOBEC, POLE, or MMR signatures.

Deciphering mutational signatures in cancer provides insight into the biological mechanisms involved in carcinogenesis and normal somatic mutagenesis. Mutational signatures have shown their applicability in cancer treatment and cancer prevention. Advances in the fields of oncogenomics have enabled the development and use of molecularly targeted therapy. More recently, mutational signatures were shown to be associated with therapeutic response and hence can serve as biomarkers to predict response to antineoplastic drugs.

Despite these promising developments, the use of mutational signatures in clinical settings is limited. The primary reason is that detecting signatures requires analyzing the mutational landscape across the whole genome, because mutational processes are not gene-specific, but rather context-dependent. In addition, a sufficient number of mutations is required to identify mutation signatures. Therefore, typically, whole-genome sequencing (WGS) is considered to be the gold standard for signatures frameworks, although whole-exome sequencing (WES) may be sufficient in some cases. However, currently, WGS and WES are mainly used for research purposes.

Conversely, targeted gene panels are routinely performed for diagnosed cancer patients, resulting in millions of targeted gene panels per year. However, targeted gene panels cover up to 2 Mb (<0.1%) of the genome, and thus can capture only a small fraction of the mutational landscape, resulting in a low count of mutations per sample.

Therefore, the ability to identify mutational signatures in targeted gene panels remains an unmet need.

The foregoing examples of the related art and limitations related therewith are intended to be illustrative and not exclusive. Other limitations of the related art will become apparent to those of skill in the art upon a reading of the specification and a study of the figures.

### SUMMARY OF INVENTION

The following embodiments and aspects thereof are described and illustrated in conjunction with systems, tools and methods which are meant to be exemplary and illustrative, not limiting in scope.

There is provided, in an embodiment, a computer-implemented method comprising: receiving, as input, a plurality of DNA sequencing samples corresponding to a cohort of subjects having various tumor types; annotating each of the DNA sequencing samples with annotations indicating (a) a tumor type associated with the DNA sequencing sample, (b) one or more mutations represented in the DNA sequencing sample, and (c) at least one dominant mutational signature associated with the DNA sequencing sample; at a training stage, training a machine learning model on a training dataset comprising: (i) the DNA sequencing samples, and (ii) labels indicating the annotations; and at an inference stage, applying the trained machine learning model to a target DNA sequencing sample from a target subject, to predict a dominant mutational signature associated with the target DNA sequencing sample.

There is also provided, in an embodiment, a system comprising at least one hardware processor; and a non-transitory computer-readable storage medium having stored thereon program instructions, the program instructions executable by the at least one hardware processor to: receive, as input, a plurality of DNA sequencing samples corresponding to a cohort of subjects having various tumor types, annotate each of the DNA sequencing samples with annotations indicating (a) a tumor type associated with the DNA sequencing sample, (b) one or more mutations represented in the DNA sequencing sample, and (c) at least one dominant mutational signature associated with the DNA sequencing sample, at a training stage, train a machine learning model on a training dataset comprising: (i) the DNA sequencing samples, and (ii) labels indicating the annotations, and at an inference stage, apply the trained machine learning model to a target DNA sequencing sample from a target subject, to predict a dominant mutational signature associated with the target DNA sequencing sample.

There is further provided, in an embodiment, a computer program product comprising a non-transitory computer-readable storage medium having program instructions embodied therewith, the program instructions executable by at least one hardware processor to: receive, as input, a plurality of DNA sequencing samples corresponding to a cohort of subjects having various tumor types; annotate each of the DNA sequencing samples with annotations indicating (a) a tumor type associated with the DNA sequencing sample, (b) one or more mutations represented in the DNA sequencing sample, and (c) at least one dominant mutational signature associated with the DNA sequencing sample; at a training stage, train a machine learning model on a training dataset comprising: (i) the DNA sequencing samples, and (ii) labels indicating the annotations; and at an inference stage, apply the trained machine learning model to a target DNA sequencing sample from a target subject, to predict a dominant mutational signature associated with the target DNA sequencing sample.

In some embodiments, the training stage comprises pre-learning, by the machine learning model, of embeddings within an n-dimensional vector space, for each of the dominant mutational signatures and the mutations represented in the DNA sequencing samples.

In some embodiments, the inference stage comprises: (i) creating, for each mutation represented in the target DNA sequencing sample, a vector embedding within the n-dimensional vector space; (ii) calculating similarity scores between (a) a mean mutational embedding vector of all of the vector embeddings created for each mutation represented in the target DNA sequencing sample, and (b) each of the dominant mutational signature embeddings pre-learned during the training stage; and (iii) selecting the pre-learned dominant mutational signature embedding representing the highest the similarity score, as the predicted dominant mutational signature associated with the target DNA sequencing sample.

In some embodiments, the similarity score is based on a cosine similarity calculation.

In some embodiments, the target DNA sequencing sample comprises between 1-15 mutations.

In some embodiments, the annotations further indicate, for at least some of the DNA sequencing samples, at least one of the following categories of annotations: tumor site, metastatic site, structural variants, smoking history of the corresponding subject, vital status of the corresponding subject, survival status of the corresponding subject, sex of the corresponding subject, or age of the corresponding subject.

In some embodiments, the annotations with respect to the mutations represented in the DNA sequencing samples are indicated by their 5-mer sequence.

In some embodiments, with respect to at least some of the DNA sequencing samples, the annotations indicate a combination of two dominant mutational signatures.

In some embodiments, the training stage further comprises pre-learning joint embeddings for each of the combinations of two dominant mutational signatures.

In some embodiments, the inference stage comprises: (i) creating, for each mutation represented in the target DNA sequencing sample, a vector embedding within the n-dimensional vector space; (ii) calculating similarity scores between (a) a mean mutational embedding vector of all of the vector embeddings created for each mutation represented in the target DNA sequencing sample, and (b) each of the pre-learned dominant mutational signature embeddings and the joint embeddings; and (iii) selecting (a) the pre-learned dominant mutational signature embedding or (b) the joint embedding, which represents the highest the similarity score, as the predicted dominant mutational signature associated with the target DNA sequencing sample.

There is further provided, in an embodiment, a method of treating cancer in a patient in need thereof, the method comprising the steps of: providing a DNA sequencing sample from the patient; applying the computer-implemented method of any one of claims 1-10 for predicting a dominant mutational signature associated with the DNA sequencing sample; and administering a cancer treatment to the patient, based, at least in part, on the predicted dominant mutational signature.

There is further provided, in an embodiment, a method of predicting survival in a cancer patient, the method comprising the steps of: providing a DNA sequencing sample from the patient; applying the computer-implemented method of any one of claims 1-10 for predicting a dominant mutational signature associated with the DNA sequencing sample; and predicting survival in the patient based, at least in part, on the predicted dominant mutational signature.

There is further provided, in an embodiment, a method of predicting immunotherapy response in a cancer patient, the method comprising the steps of: providing a DNA sequencing sample from the patient; applying the computer-implemented method of any one of claims 1-10 for predicting a dominant mutational signature associated with the DNA sequencing sample; and predicting immunotherapy response in the patient based, at least in part, on the predicted dominant mutational signature.

There is further provided, in an embodiment, a method of treating cancer in a patient in need thereof, the method comprising the steps of: providing a DNA sequencing sample from the patient; applying, to the DNA sequencing sample, a machine learning model trained to predict a dominant mutational signature associated with the DNA sequencing sample; and administering a cancer treatment to the patient, based, at least in part, on a dominant mutational signature in the DNA sequencing sample predicted by the application.

There is further provided, in an embodiment, a method of predicting survival in a cancer patient, the method comprising the steps of: providing a DNA sequencing sample from the patient; applying, to the DNA sequencing sample, a machine learning model trained to predict a dominant mutational signature associated with the DNA sequencing sample; and predicting survival in the patient based, at least in part, on a dominant mutational signature in the DNA sequencing sample predicted by the application.

There is further provided, in an embodiment, a method of predicting immunotherapy response in a cancer patient, the method comprising the steps of: providing a DNA sequencing sample from the patient; applying, to the DNA sequencing sample, a machine learning model trained to predict a dominant mutational signature associated with the DNA sequencing sample; and predicting immunotherapy response in the patient based, at least in part, on a dominant mutational signature in the DNA sequencing sample predicted by the application.

In addition to the exemplary aspects and embodiments described above, further aspects and embodiments will become apparent by reference to the figures and by study of the following detailed description.

### BRIEF DESCRIPTION OF THE FIGURES

FIG. 1A illustrates an exemplary training workflow of the present machine learning model, in accordance with some embodiments of the present invention;

FIG. 1B shows a visualization of signature embeddings created by the training process exemplified in FIG. 1A, in accordance with some embodiments of the present invention;

FIG. 1C illustrates an exemplary inferencing process of a trained machine learning model of the present disclosure, in accordance with some embodiments of the present invention;

FIG. 2 is a block diagram of an exemplary system which provides for training a machine learning model configured to identify specific mutational signatures based on a limited number of input mutations, in accordance with some embodiments of the present invention;

FIG. 3 illustrates the functional steps in a method for training a machine learning model configured to identify a dominant mutational signature in a DNA sequencing sample obtained from a subject, in accordance with some embodiments of the present invention;

FIGS. 4A-4D show experimental results of the present machine learning model in predicting mutational signature with down-sampled target samples, in accordance with some embodiments of the present invention;

FIGS. 5A-5B show experimental results of the present machine learning model in predicting mutational signatures in PCAWG WGS Samples, in accordance with some embodiments of the present invention;

FIGS. 6A-6H show experimental results of the present machine learning model in predicting mutational signatures in MSK-IMPACT cohort, in accordance with some embodiments of the present invention;

FIGS. 7A-7F show experimental clinical results of the present machine learning model in predicting mutational signatures in the MSK-ICI and MSK-MET cohorts, in accordance with some embodiments of the present invention;

FIGS. 8A-8B show experimental clinical results of the present machine learning model in predicting mutational signatures in the MSK-ICI and MSK-MET cohorts, in accordance with some embodiments of the present invention;

FIGS. 9A-9B shows a comparison of p-values and hazard ratio values between results of multivariate survival analysis (as presented in FIGS. 7A-7F and 8A-8B), in accordance with some embodiments of the present invention;

FIGS. 10A-10E show survival rates of NSCLC patients, in accordance with some embodiments of the present invention; and

FIGS. 11A-11B show the interpretability of mutations and the UV signature embeddings representations, in accordance with some embodiments of the present invention.

### DETAILED DESCRIPTION

Disclosed is a technique, embodied in a system, computer-implemented method, and computer program product, which provides for a machine learning model trained to identify a dominant mutational signature in a DNA sequencing sample obtained from a subject. In some embodiments, a machine learning model of the present disclosure may be trained to identify a dominant mutational signature in a DNA sequencing sample obtained from a subject, wherein the DNA sequencing sample may consist of only a limited amount of sequencing data, for example, a sequencing sample obtained using a targeted gene panel.

As used herein, the terms ‘mutational signatures’ or ‘signatures’ generally refer to patterns of mutations characteristic of a mutagenesis process.

In some embodiments, the present machine learning model learns, at a training stage, embeddings representative of mutations and mutational signatures associated with various tumor types, as well as the contextual relationships between the mutations and their signatures. In some embodiments, the trained machine learning model may then be applied, at an inference stage, to a target sequencing sample obtained from a subject, to predict a dominant signature associated with the target sample. In some embodiments, the prediction is based, at least in part, on the individual mutations present in the target sample. In some embodiments, the target sample may consist of only a limited amount of sequencing data, e.g., a limited number of mutations.

In some embodiments, the present machine learning model learns contexts of mutations and signatures based on a training dataset which comprises a plurality of tumor sequencing samples obtained from a cohort of subjects, wherein each sample is annotated with annotations indicating at least some of the following:
The type of the tumor,
the classes of mutations represented in the sample (considering, for each mutation, its penta-nucleotide context, e.g., the mutational event and 2 bases-5′ and 3′, for a total of 1536 possible options), and
a dominant signature associated with the sample, such as APOBEC (SBS2, SBS13), UV (SBS7a-d and SBS38), Tobacco (SBS4), HRD (SBS3), MMR (SBSs 6, 14, 15, 20, 21, 26, and 44), POLE (SBS10a-b), Clock_SBS1, and Clock_SBS5.

In some embodiments, the present machine learning model incorporates natural language processing (NLP) machine learning techniques, based on the insight that a DNA sequencing sample may be viewed as a document, comprising individual mutations which may be likened to words, or tokens, while the dominant mutational signature active in the sample and the cancer type associated therewith may be likened to ‘tags’ assigned to the document as a whole. The aim of the present machine learning model is, therefore, to create, during training, numerical representations (or embeddings) for each DNA training sample based on its individual mutations and its assigned ‘tags’ of dominant signature and associated tumor type, which maximize the similarities between related features (for example, mutations caused by UV damage and the UV signature embeddings) and simultaneously minimize similarities with unrelated features (for example, UV signature and Tobacco signature embeddings).

In some embodiments, the trained machine learning model of the present disclosure may be inferenced on a target sample, to create a target vectorial embedding representation. The target embedding may then be compared to the signatures embeddings created during training, to predict whether it is represented among the learned embeddings.

The present inventors conducted experiments which show the robustness of the predictions of the present machine learning model in various settings, including when inferenced on WGS samples, down-sampled WES (down to 20% of the mutational load or down to 1-15 mutations), and targeted gene panels with high tumor mutational burden.

A potential advantage of the present invention is, therefore, in that it may accurately predict associations between specific mutations and mutational signatures based on a sample comprising only a relatively low mutation count, e.g., as low as four mutations, or even a single mutation in the case of certain signatures. Thus, the present invention may have significant clinical implications in situations where only a limited amount of DNA sequencing data is available, as in the case of common clinical assays, such as targeted gene panels. The present invention provides an easy-to-use way to detect a dominant signature in clinical-setting assays with high accuracy, which may lead in turn to better personalized medicine for cancer patients.

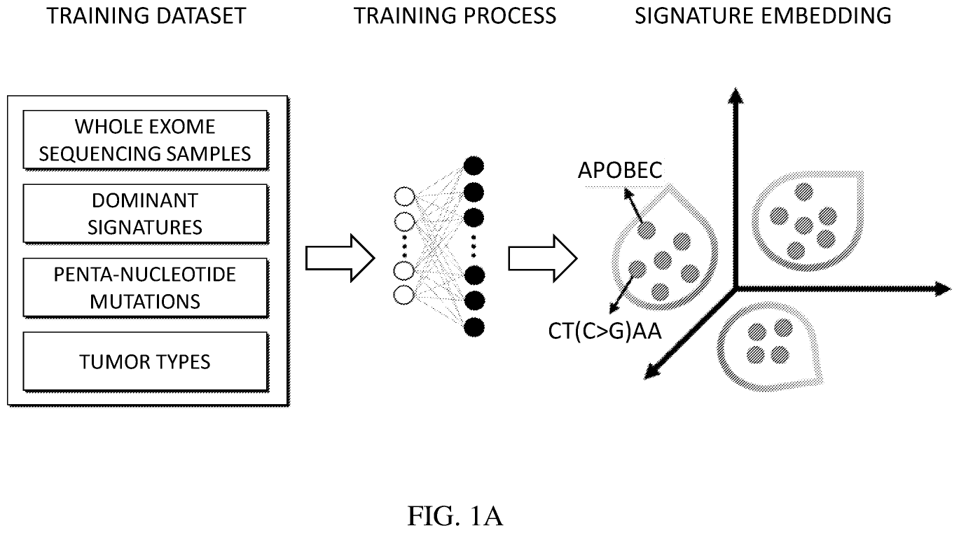

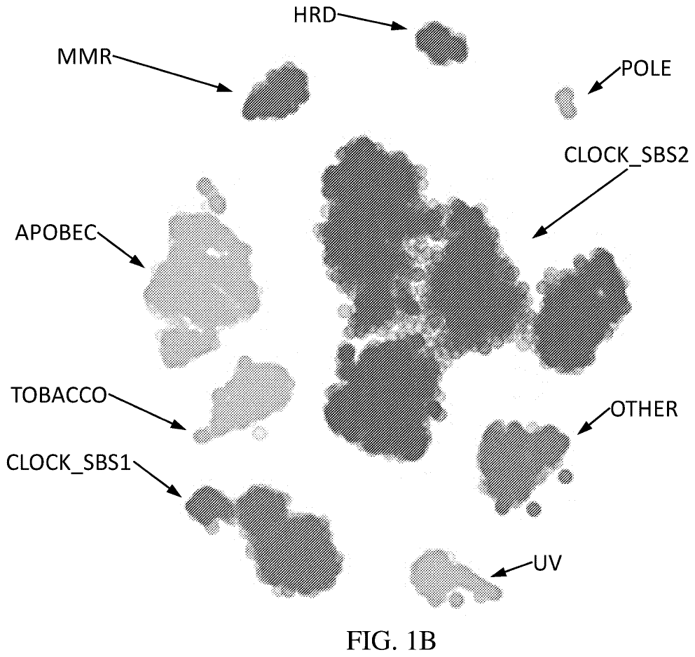

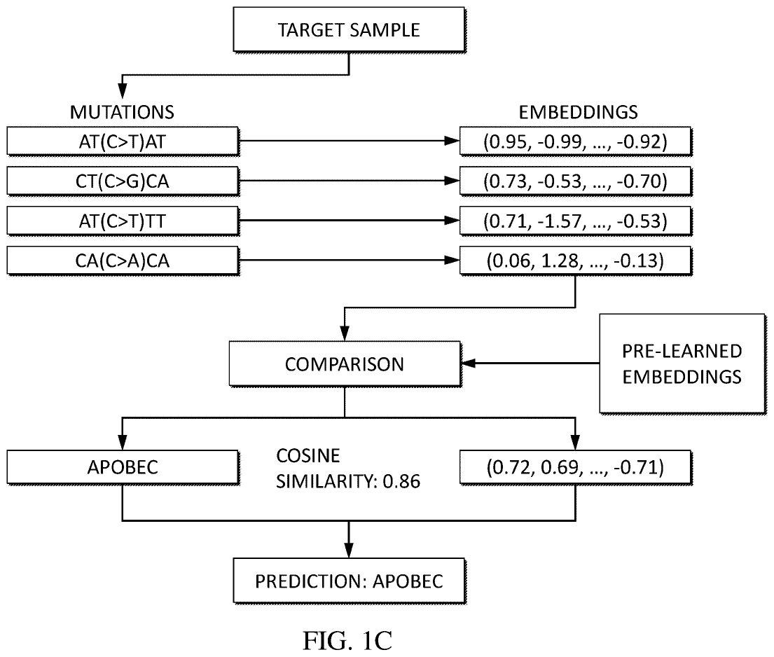

FIG. 1A illustrates an exemplary training workflow of the present machine learning model. The model is trained on a training dataset (left), which comprises sequencing samples of tumors, with annotations indicating the mutations represented therein, and the dominant signature and tumor type associated therewith. The model's training process (middle) aims to decrease a loss while maximizing similarities between related features and minimizing similarities between unrelated features are. The result is a set of embeddings of the various signatures and related mutations within a cartesian coordinate system (right).

FIG. 1B shows a visualization of signature embeddings created by the training process exemplified in FIG. 1A, reduced to two-dimensions by applying a t-distributed stochastic neighbor embedding (t-SNE projections) algorithm. As can be seen, the signature embeddings are distinctly clustered in the visualized embedding space.

FIG. 1C illustrates an exemplary inferencing process of a trained machine learning model of the present disclosure. Embedding representations are created for a target sample which contains, in this example, 4 mutations. Then, a cosine similarity between the created target profile and each of the signature embeddings created during trainings is calculated. The embedding having the highest cosine similarity to the target profile embedding (provided the cosine is greater than a predetermined threshold, e.g., 0.6), is predicted as the dominant signature active in the target sample.

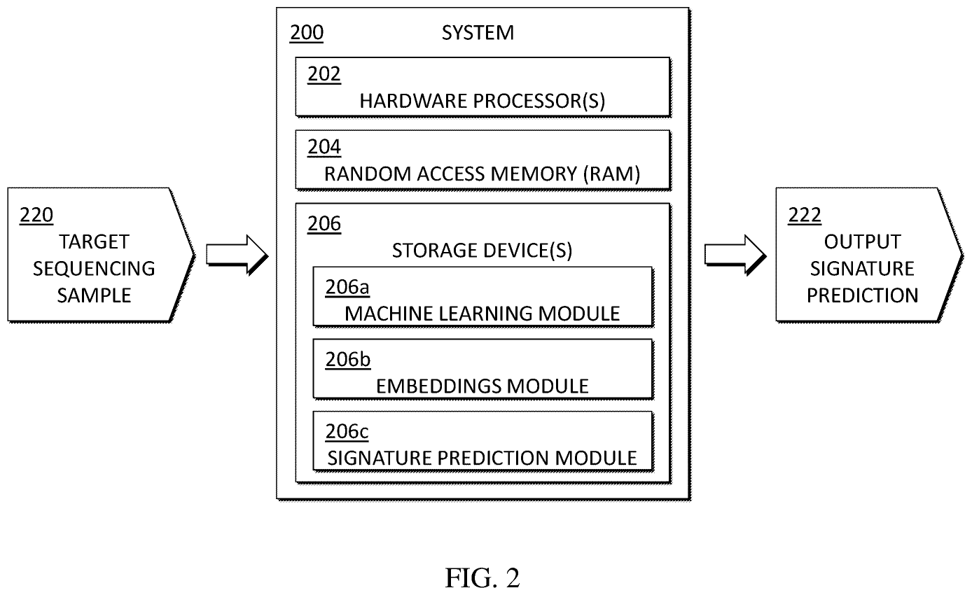

FIG. 2 is a block diagram of an exemplary system 200 which provides for training a machine learning model configured to identify specific mutational signatures based on a limited number of input mutations, in accordance with some embodiments of the present invention.

In some embodiments, system 200 may comprise a hardware processor 202, and a random-access memory (RAM) 204, and/or one or more non-transitory computer-readable storage device 206. In some embodiments, system 200 may store in storage device 206 software instructions or components configured to operate a processing unit (also ‘hardware processor,’ ‘CPU,’ ‘quantum computer processor,’ or simply ‘processor’), such as hardware processor 202. In some embodiments, the software components may include an operating system, including various software components and/or drivers for controlling and managing general system tasks (e.g., memory management, storage device control, power management, etc.) and facilitating communication between various hardware and software components. Components of system 200 may be co-located or distributed, or the system may be configured to run as one or more cloud computing ‘instances,’ ‘containers,’ ‘virtual machines,’ or other types of encapsulated software applications, as known in the art.

Storage device(s) 206 may have stored thereon program instructions and/or components configured to operate hardware processor(s) 202. The program instructions may include one or more software modules, such as a machine learning module 206a, an embeddings module 206b, and a signature prediction module 206c.

Machine learning module 206a may comprise any one or more suitable neural networks architectures (i.e., which include one or more neural network layers), and can be implemented using any suitable optimization algorithm. In some embodiments, machine learning module 206a may be configured to train a machine learning model of the present disclosure to identify a dominant mutational signature in a DNA sequencing target sample 220 obtained from a subject.

In some embodiments, machine learning module 206a may employ any one or more natural language processing (NLP) algorithms and/or clustering algorithms, such as genetic clustering algorithms, Bayesian clustering, C-means, K-means, Fuzzy logic, or the like.

In some embodiments, a machine learning model trained by machine learning module 206a may be configured to perform a clustering operation on DNA sequencing sample input data, to pre-learn embeddings of individual mutations based on their associated mutational signatures. In some embodiments, a machine learning model trained by machine learning module 206a may incorporate an embeddings module 206b configured to generate token embeddings with respect to each mutation identified in an input sequencing sample, which maps each mutation onto an n-dimensional vector space, based on its context. Thus, mutations that have similar contexts will appear roughly in the same area of the vector space. Each mutation in the input sequencing samples is assigned a numerical vector which represents the mutation within the embedding space, where the embedding space itself captures the relationships between the various mutations represented in the input data and their associated mutational signatures and tumor types. The embedding process generally assigns vector representations that are close together in the embedding space to mutations having similar mutational signature contexts. Once the input data is represented numerically within the embedding space, it can be used in any mathematical, statistical, or machine learning operations and analyses, for example, to generate classifications and predictions with respect to unseen data.

Accordingly, in some embodiments, a machine learning model trained by machine learning module 206a may be configured to use embeddings module 206b to pre-learn mutation embeddings, based on input data comprising a plurality of DNA sequencing samples, wherein mutations, mutational signatures, and tumor types are indicated for each input sample. The pre-learning process thus learns general representation, which can then be used to predict the contextual mutational signatures of unseen target sequencing samples.

In some embodiments, signature prediction module 206c may comprise one or more algorithms for formulating and outputting a prediction 222 with respect to a mutational signature represented in the target DNA sequencing sample, based on inferencing a trained machine learning model of the present disclosure.

System 200 as described herein is only an exemplary embodiment of the present invention, and in practice may be implemented in hardware only, software only, or a combination of both hardware and software. System 200 may have more or fewer components and modules than shown, may combine two or more of the components, or may have a different configuration or arrangement of the components. System 200 may include any additional component enabling it to function as an operable computer system, such as a motherboard, data busses, power supply, a network interface card, a display, an input device (e.g., keyboard, pointing device, touch-sensitive display), etc. (not shown). Moreover, components of system 200 may be co-located or distributed, or the system may be configured to run as one or more cloud computing ‘instances,’ ‘containers,’ ‘virtual machines,’ or other types of encapsulated software applications, as known in the art.

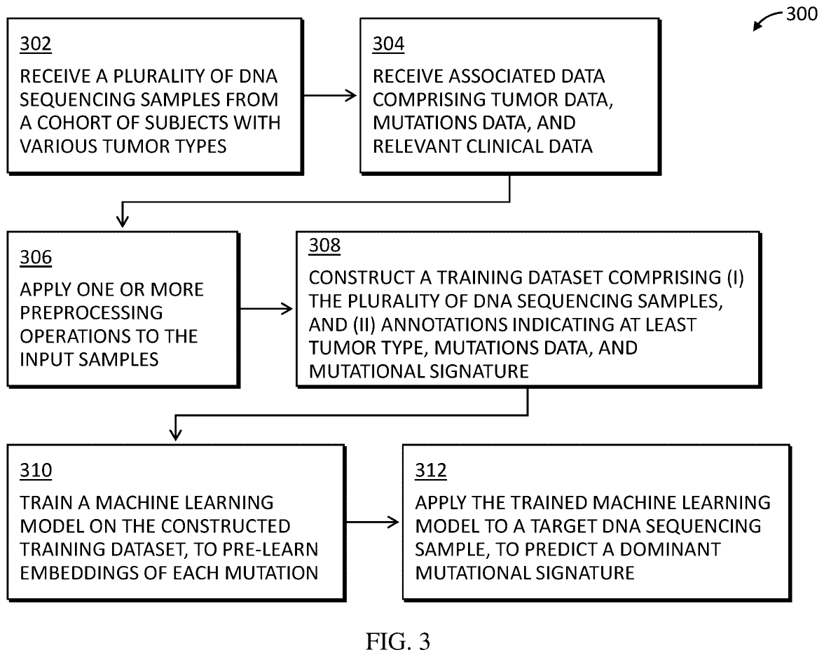

The instructions of system 200 will now be discussed with reference to the flowchart of FIG. 3, which illustrates the functional steps in a method 300 for training and inferencing a machine learning model configured to identify a dominant mutational signature in a DNA sequencing sample obtained from a subject, in accordance with some embodiments of the present invention.

The various steps of method 300 will be described with continuous reference to exemplary system 200 shown in FIG. 2. The various steps of method 300 may either be performed in the order they are presented or in a different order (or even in parallel), as long as the order allows for a necessary input to a certain step to be obtained from an output of an earlier step. In addition, the steps of method 300 may be performed automatically (e.g., by system 200 of FIG. 2), unless specifically stated otherwise. In addition, the steps of method 300 are set forth for exemplary purposes, and it is expected that modifications to the flowchart may be implemented as necessary or desirable.

Method 300 begins at step 302, wherein the instructions of machine learning module 206a may cause system 200 to receive, as input, a plurality of DNA sequencing samples obtained from a cohort of subjects with a variety of tumor types. In some embodiments, the input sequencing samples may comprise whole exome sequencing (WES) and/or whole genome sequencing (WGS) mutational profiles.

An exemplary implementation of step 302 by the present inventors used DNA sequencing samples obtained from one or more of the following sources:
The Cancer Genome Atlas (TCGA) project: TCGA catalogues the genetic mutations responsible for cancer using genome sequencing and bioinformatics. (https://www.cancer.gov/about-nci/organization/ccg/research/structural-genomics/tcga).
The Memorial Sloan Kettering-Integrated Mutation Profiling of Actionable Cancer Targets (MSK-IMPACT): This dataset includes mutation profiling of 10,000 sequencing cancer-related samples. (https://www.mskcc.org/msk-impact).
The Memorial Sloan Kettering—Metastatic Events and Tropisms (MSK-MET): This dataset includes samples from a pan-cancer cohort of tumor genomic and clinical outcome data from 25,000 patients. (https://www.cbioportal.org/study/summary?id=msk_met_2021).
The Memorial Sloan Kettering—Immune Checkpoint Inhibitor (MSK-ICI): See, R. M. Samstein, et al. Tumor mutational load predicts survival after immunotherapy across multiple cancer types. Nature Genetics. 51, 202-206 (2019).
GENIE: American Association for Cancer Research (AACR) Project GENIE, which comprises more than 154,000 sequenced cancer samples from 137,000 patients (see, Powering Precision Medicine through an International Consortium. Cancer Discovery. 7, 818-831 (2017)).

In step 304, the instructions of machine learning module 206a may cause system 200 to receive, as input, data with respect to at least some of the input sequencing samples received in step 302, and to associate these input data with their corresponding input sequencing samples. In some embodiments, the input data received in step 304 comprises, per input sequencing sample, at least one of:
A tumor type associated with the input sequencing sample.
Individual mutations represented in the input sequencing sample.
Dominant mutational signature associated with the input sequencing sample.

In some embodiments, the input data received in step 304 comprises one or more of the following additional indications per input sequencing sample:
Additional cancer and tumor details.
Tumor site.
Metastatic site.
Structural variants.
Clinical history of subject:
   smoking history.
   Vital status.
   Survival status.
   Somatic status.
Demographic data of subject:
   Sex.
   Age.

In some embodiments, individual mutations represented in an input sequencing sample may be indicated by their 5-mer sequence, using the conventional classification based on the six substitution subtypes, and the 2 immediate 5′ and 3′ nucleotides to the mutation. Overall, there are 6·(4⁴)=1,536 possible mutations classifications. It is noted that the relatively lower number of individual mutations introduces sparsity into the pre-learning process, which allows more specificity for each mutation's representation, and thus for increased classification performance.

In some embodiments, dominant mutational signature indications per input sequencing sample may be based on publicly-available information (see, for example, L. B. Alexandrov, et al. The repertoire of mutational signatures in human cancer. Nature. 578, 94-101 (2020); A. Zehir, et al. Mutational landscape of metastatic cancer revealed from prospective clinical sequencing of 10,000 patients. Nature Medicine. 23, 703-713 (2017)).

In some embodiments, the indication of a dominant mutational signature in each input sequencing sample is based, at least in part, on a relative contribution value of the mutational signature in the input sequencing sample, for example, the proportion of mutations in an input sequencing sample out of the total number of mutations that are associated with a particular signature. This is because there may be more than one mutational signature active in each input sequencing sample, with different degrees of prominence or dominance.

In one implementation, the present inventors assigned mutational signature indications per input sequencing samples, based on the following methodology:
Mutational signature: APOBEC
   Mutations: SBS2+SBS13
   Relative contribution: >30%
Mutational signature: UV
   Mutations: SBS7a-d and SBS38
   Relative contribution: >30%
Mutational signature: Tobacco
   Mutations: SBS4
   Relative contribution: >30%
Mutational signature: HRD
   Mutations: SBS3
   Relative contribution: >50%
Mutational signature: Clock_SBS1
   Mutations: SBS1;
   Relative contribution: >40%
Mutational signature: Clock_SBS5
   Mutations: SBS5
   Relative contribution: >40%
Mutational signature: POLE
   Mutations: SBS10a-b
   Relative contribution: >20%
Mutational signature: MMR
   Mutations: SBS6, SBS14, SBS15, SBS20, SBS21, SBS26, SBS44
   Relative contribution: >30%

As can be seen, in this particular implementation, in order for an input sequencing sample to be annotated with one of the signatures APOBEC, Tobacco, UV or MMR, a relative contribution of at least 30% of the corresponding signature is required. In order for a sample to be annotated with the signature HRD, a relative contribution of at least 50% contribution is required, because the mutation SBS3 has a featureless, ‘flat,’ profile which may lead to higher rate of false-positive detection rates. In order for an input sequencing sample to be annotated with the signature POLE, a threshold of 20% is required, because POLE has a unique mutational profile with specific features, and it is usually followed by a hypermutated or an ultra-hypermutated phenotype. Because of their ubiquity, in order for an input sequencing sample to be annotated with the signatures Clock_SBS1 or Clock_SBS5, a relative contribution of at least 40% of SBS1 or SBS5, respectively, is required, and in addition, no other signature may be associated with the input sequencing sample.

In other implementations, different, additional, or fewer mutational signature classes may be used for annotating the input sample data, and different annotation methodology may be applied to the selected mutational signature classes.

With reference back to FIG. 3, in step 306, a data preprocessing stage may be performed, to apply any one or more of data cleaning, data normalizing, removal of missing data, data quality control, and/or any other suitable preprocessing method or technique.

For example, in some embodiments, data preprocessing may include removal of input sequencing samples received in step 302, based on accuracy level (as provided by the PCAWG analyses, e.g., removal of samples having accuracy of less than 0.7) and/or mutation count (e.g., removal of WES samples having fewer than 20 mutations, or WGS samples having fewer than 100 mutations).

In some embodiments, data cleaning may include removal of duplicate samples received from two or more different sources. In some embodiments, data cleaning may also include removal of samples associated with specific mutational signatures which are sparsely represented, or may be the result of sequencing artefacts, such as SBS27 and SBS45 (SBS27 is frequent in AML samples, while SBS45 is frequent in many cancer types).

With reference back to FIG. 3, in step 308, the instructions of machine learning module 206a may cause system 200 to construct a training dataset comprising:
(i) The input sequencing samples received in step 302, and
(ii) labels indicating at least a tumor type, mutations data/class, and at least one dominant mutational signature, associated with each of the input sequencing samples.

In one implementation, the mutational signature annotation labels include the following 9 signatures, based on the indication methodology detailed herein above:
APOBEC
UV
Tobacco
HRD
Clock_SBS1
Clock_SBS5
POLE
MMR
Other (for samples in which none of the previous signatures was dominant).

However, in other embodiments, mutational signature annotations may be based on any desired selected set of specific mutational signatures, comprising, e.g., 1, 2, 3, 4, 5, 6, 7, 8, 9, 10, 15, 20, or more mutational signatures.

In one implementation, input sequencing samples may be annotated or labelled with a combination of two or more dominant mutational signatures. For example, such combinations may include the signatures SBS5 or SBS1 (which are ubiquitous and affect many samples) with other signatures, such as Tobacco, UV, APOBEC, MMR, and/or POLE.

In some embodiments, the labels may additionally indicate one or more of:
Tumor site.
Metastatic site.
Structural variants.
Clinical history of subject:
   Smoking history.
   Vital status.
   Survival status.
   Somatic status.
Demographic data of subject:
   Sex.
   Age.

In some embodiments, in step 310, the instructions of machine learning module 206a may cause system 200 to train a machine learning model of the present disclosure on the training dataset constructed in step 308.

In one implementation, the instructions of embeddings module 206b may cause system 200 to pre-learn, at training stage 310, embeddings of mutations within a relevant vector space, based on the training dataset constructed in step 308, wherein at least mutations data/class, mutational signatures, and tumor types are indicated for each input sample.

In some embodiments, the instructions of embeddings module 206b may cause system 200 to take, as input, the training dataset constructed in step 308, wherein each mutation is considered a token, and wherein associated dominant signature and tumor type are considered as tags. The instructions of embeddings module 206b may cause system 200 to generate positive pair associations for each token and its associated tags.

For example, the mutation CT[C>G]AA in a sample from a bladder cancer patient with APOBEC mutational signature, would have positive pairs of (CT[C>G]AA, bladder cancer) and (CT[C>G]AA, APOBEC signature). For each such positive pair, the instructions of embeddings module 206b may cause system 200 to generate k negative pairs, which consist of tags that are not related to the mutation. For the above example, negative pairs for the relevant sample comprise (CT[C>G]AA, colorectal cancer) and (CT[C>G]AA, MMR signature). The negative pairings are generated using a k-negative random sampling strategy.

In some embodiments, the pre-learning training stage operates a neural embedding architecture, comprising an input layer and output layer. Each forward pass consists of calculating the dot product as given by:

$$dot ( A , B ) = {\sum }_{i = 1}^{n} {A}_{i} {B}_{i}$$

where A and B stand for embedding vectors for all positive pairs (i.e., embeddings of different entities of the same sample) and k negative pairs. These dot product scores (positive and negative scores) are then combined to calculate a hinge loss function, as follows:

$$L = \sum ( dot ( a , {b}^{+} ) , dot ( a , {b}_{1}^{-} ) , … , dot ( a , {b}_{k}^{-} ) )$$

where b⁺ and b⁻ represent positive pairs and negative pairs, respectively. In each epoch, the loss may be optimized using any suitable algorithms, such as the Decoupled Weight Decay Adam algorithm. Hyperparameters included are the length of the embedding vector (l), number of negative samples per positive sample (k), embedding parameter (p), and hinge loss constant margin (u). In one implementation, the following hyperparameter values were used: l=200, k=30, p=0.5, and u=1, with a learning rate of 1e−3, 20 epochs, a batch size of 4,096, and a seed set to 88.

In some embodiments, step 310 comprises creating vector embeddings associated with two or more dominant mutational signatures. In such cases, the mean embedding vectors of two or more individual mutational signatures generated in step 310, may be used to create a new embedding vector representing the two or more mutational signatures.

In one implementation, step 310 may comprise creating vector embeddings associated with two or more dominant mutational signatures, for the respective combinations of the signatures SBS5 and SBS1 (which are ubiquitous and affect many samples) with other signatures. Thus, for example, the mean embedding vectors of Clock_SBS5 and Tobacco may be used to create a joint embedding vector, Tobacco_SBS5, representing the combination of Tobacco and SBS5 together. During inference (which will be discussed in the following step 312) the joint vector embedding Tobacco_SBS5 may be compared to the mutational embedding vector of a tumor sample. Then, if the cosine similarity of the sample embedding to the joint embedding Tobacco_SBS5 is greater than the respective similarity scores associated with Clock_SBS5 and Tobacco individually, it may be concluded that the particular sample carries both signatures.

In some embodiments, the present technique may generate, in step 310, joint mutational signature embeddings for at least one or more of the following mutational signature combinations:
UV_SBS5
UV_SBS1
APOBEC_SBS5
APOBEC_SBS1
MMR_SBS5
MMR_SBS1
POLE_SBS5
POLE_SBS1
TOBACCO_SBS5
TOBACCO_SBS1

With reference back to FIG. 3, in step 312, the instructions of signature prediction module 206c may cause system 200 to inference the machine learning model pre-learned and trained in step 310, on an unseen target DNA sequencing sample 220 (shown in FIG. 2) obtained from a target subject, to output a prediction 222 (shown in FIG. 2) with respect to a mutational signature associated with the target sample 220.

In some embodiments, inferencing step 312 comprises extracting the penta-nucleotide mutational landscape from sample 220, as described above. For each of the extracted mutations, an embedding vector is identified based on the pre-learning performed in step 310. The mean mutational embedding vector of sample 220 is then compared to each of the pre-learned signatures embedding (which may include joint embeddings representing two or more individual mutational signatures, such as TOBACCO_SBS5), to calculate a corresponding similarity score between the mean mutational embedding of sample 220 and each of the pre-learned mutational signatures.

In some embodiments, the similarity score in based, at least in part, on cosine similarity, as follows:

$$s {imilarity}_{c} ( A , B ) = \frac{{\sum }_{i = 1}^{n} {A}_{i} {B}_{i}}{\sqrt{{\sum }_{i = 1}^{n} {A}_{i}^{2}} \sqrt{{\sum }_{i = 1}^{n} {B}_{i}^{2}}}$$

where a cosine value of 1 means identical vectors.

In some embodiments, the instructions of signature prediction module 206c may cause system 200 to select and output the pre-learned signature embedding representing the highest similarity score with the mean mutational embedding of sample 220, as the predicted mutational signature 222 associated with sample 220.

In some embodiments, the instructions of signature prediction module 206c may cause system 200 to issue a prediction only when the highest similarity score exceeds a minimum threshold value, such as 0.60. When no similarity score exceeds the minimum threshold, the instructions of signature prediction module 206c may cause system 200 to not issue a prediction.

### Experimental Results

The present inventors conducted several experiments to validate the present machine learning model on a variety of validation datasets, as shall be further detailed below.

Validation datasets used in the experiments included DNA sequencing samples obtained from the following publicly-available datasets, or as otherwise indicated:
The Cancer Genome Atlas (TCGA) project,
Pan-cancer Analysis of Whole Genomes (PCAWG) project,
The Memorial Sloan Kettering-Integrated Mutation Profiling of Actionable Cancer Targets (MSK-IMPACT), and
The Memorial Sloan Kettering—Metastatic Events and Tropisms (MSK-MET).
MSK-ICI
GENIE.
   Predicting Mutational Signature with Down-Sampled Target Samples

To test the ability of the present trained machine learning model to correctly predict a mutational signature from a target sample containing a limited amount of sequencing data, the present inventors constructed a validation dataset comprising 2,880 TCGA samples in which the dominant signature accounts for at least 50% of mutations (except Clock_SBS5, where the minimum contribution threshold was set at 75%).

The trained machine learning model of the present disclosure was then repeatedly inferenced on random samples obtained from the validation dataset, based on random down-sampling of the number of mutations represented in each sample, using two different methodologies:
(i) Randomly selecting only 20% of the mutations represented in each sample.
(ii) Randomly selecting a set number of between 1-15 of the mutations represented in each sample.

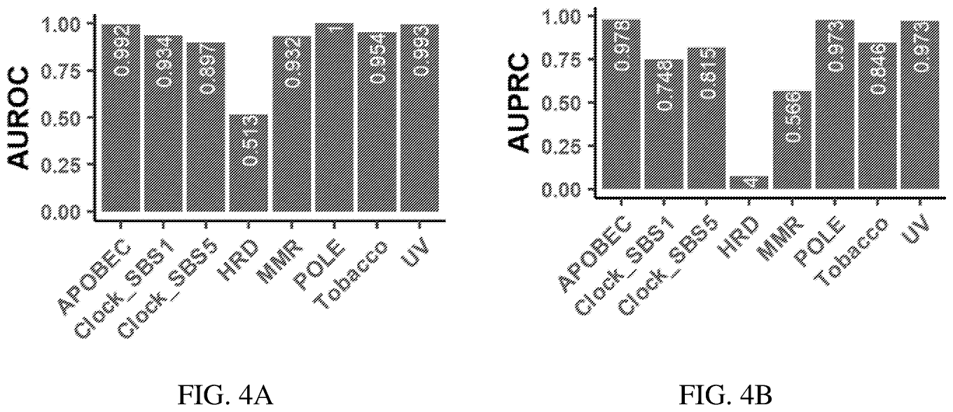

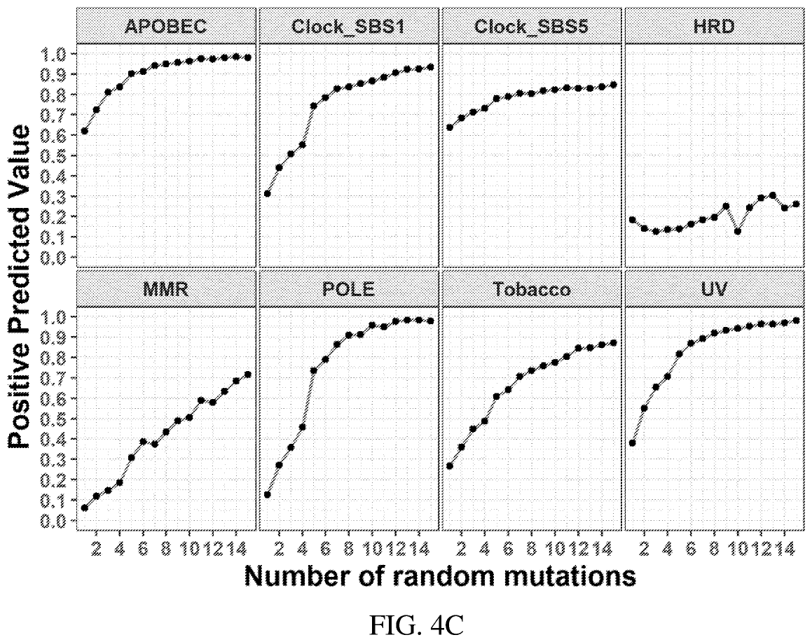

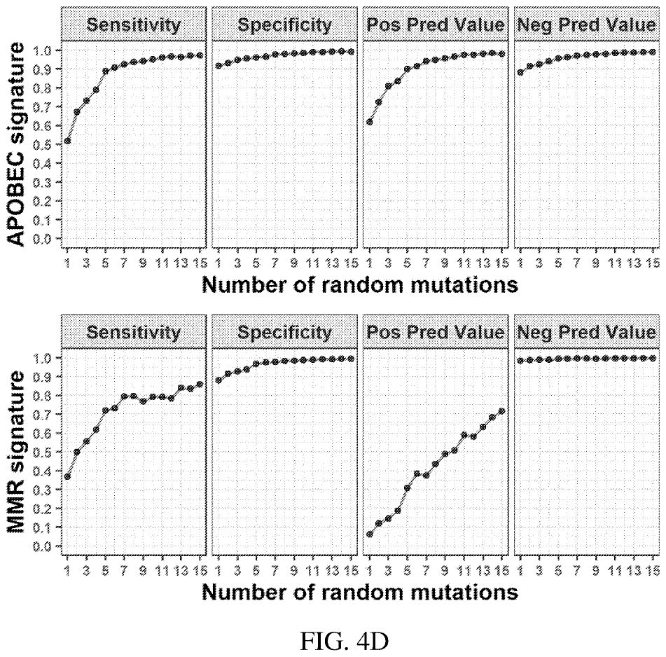

The results of these tests are presented in FIGS. 4A-4D.

FIG. 4A show the Area Under the ROC (AUROC), and FIG. 4B the Area Under the Precision-Recall Curve (AUPRC) scores for predicting the dominant signature in input samples that were down-sampled to a randomly selected 20% mutations from the validation set of 2,880 samples, as described above. Values are average across 10 iterations.

FIGS. 4C and 4D shows prediction results of the present machine learning model in input samples that were randomly down-sampled to a set number of mutations ranging from 1-15, averaged across 10 iterations for each set number of mutations. FIG. 4C shows positive predictive value (PPV) scores of different signatures. FIG. 4D shows the sensitivity, specificity, PPV, and Negative predictive value (NPV) for the APOBEC signature. As can be expected, the predictive power of the trained machine learning model depends on the number of mutations represented in the target sample, wherein the higher the number of mutations, the more accurate the prediction. However, this trend differs across the various signatures. The lowest number of mutations were needed for APOBEC and UV predictions, while for the MMR signature, a greater number of mutations is needed and the prediction scores are lower. In the case of MMR, the reason is likely due to mismatch with Clock_SBS1, as both signatures share prominent mutations of C>T at CpG.

### Predicting Signature Label in PCAWG WGS Samples

The present inventors further tested the present machine learning model, by inferencing it on an independent dataset of 2,750 PCAWG WGS samples (see, Pan-cancer analysis of whole genomes. Nature. 578, 82-93 (2020)).

The dataset comprises 2,750 samples, of which 2,117 are Clock_SBS5-dominant. First, the present machine learning model was inferenced without any modifications to the mutations represented in the samples, using the pre-learned embeddings (see step 310 with reference to FIG. 3). Compared to TCGA WES samples reported immediately above, PCAWG WGS samples comprise roughly 65 times more mutations (medians of 83 and 5260, respectively).

The PCAWG WGS dataset includes 1,253 samples, of which 929 are Clock_SBS5 and only one Clock_SBS1. A total of 0.2% of mutations per sample were randomly selected, with a minimum of 10 mutations for samples with fewer than 5,000 mutations. The trained machine learning model was inferenced on the selected mutations from each sample, to predict the dominant signature. Overall, the classification metrics were relatively high and mostly consistent with the TCGA results reported herein above. Thus, the present machine learning model was able to predict accurately both in samples with exceptionally low mutational load, and in WGS samples with as many as hundreds of thousands of mutations.

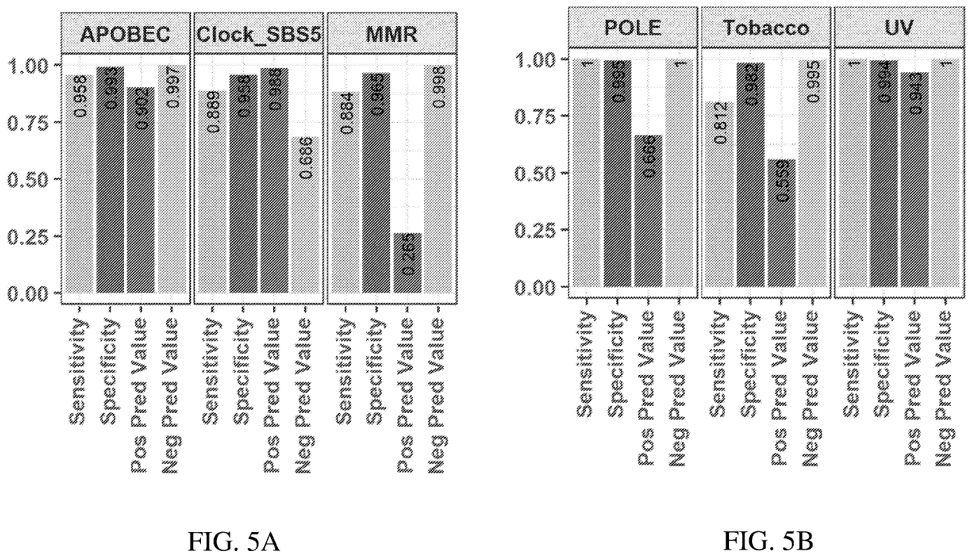

The prediction results are shown in FIGS. 5A-5B. FIGS. 5A-5B show sensitivity, specificity, PPV and NPV scores of predictions of the present machine learning model on a benchmark validation set comprising 1,153 WGS samples, down sampled to 0.2% mutations, averaged across 10 iterations. Results are shown for the APOBEC, Clock_SBS5, MMR, POLE, Tobacco, and UV signatures.

In addition, sensitivity, specificity, PPV and NPV were measured with no down sampling of mutations, for APOBEC, Clock_SBS1, Clock_SBS5, HRD, MMR, Tobacco, and UV. The present machine learning model predicted correctly PCAWG samples with high evaluation scores. Furthermore, almost all misclassifications were the result of mislabeling of Clock_SBS5-dominant samples. The reason for the mislabeling is likely the fact that SBS5 is a ubiquitous signature that is active at some level in almost any cell. The PPV scores of all signatures, except SBS1 and HRD, were 91.4%-100%. Of note, only 9 samples were labeled as Clock_SBS1 in PCAWG WGS, which may account for its relatively low performance in PCAWG predictions.

### Predicting Signatures in Targeted Gene Panels

The present inventors constructed four targeted gene panel validation datasets from the following data sources:
MSK-IMPACT
MSK-MET
MSK-ICI

### MSK-IMPACT Panel

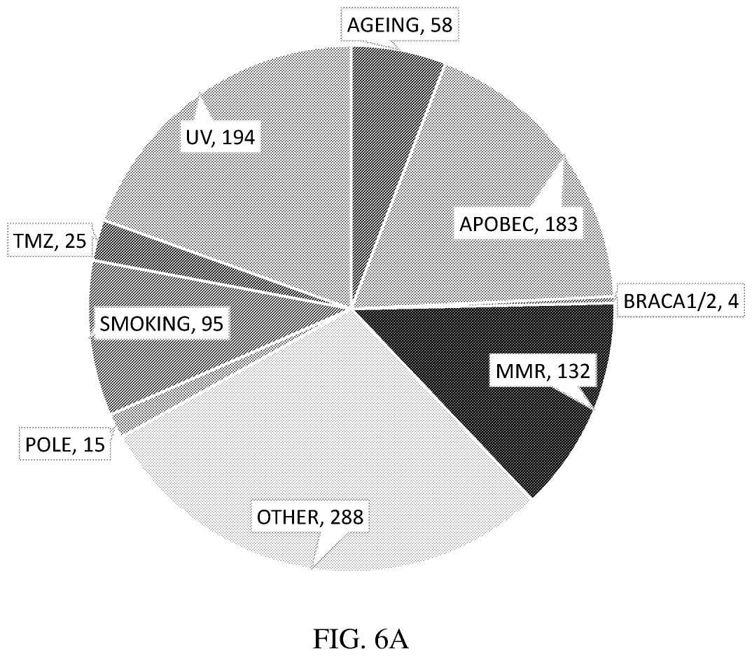

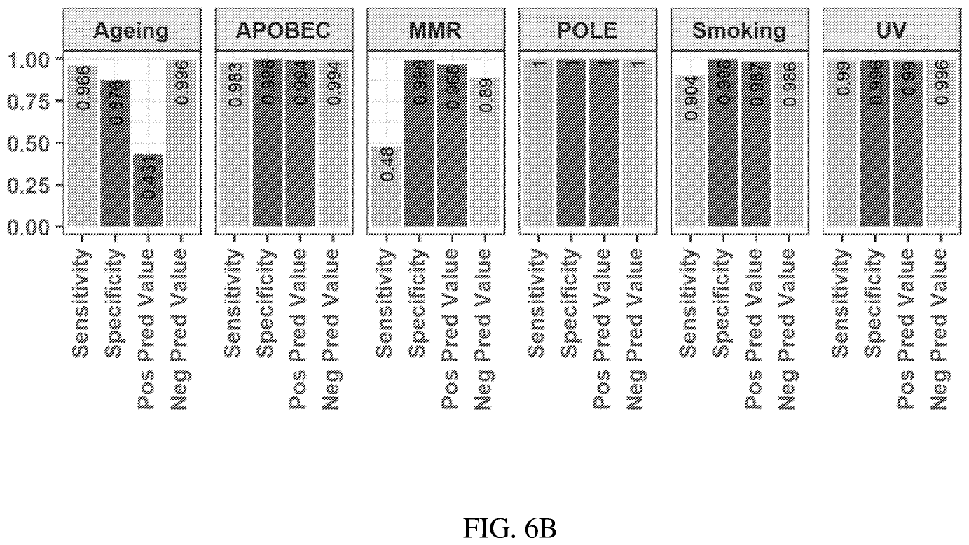

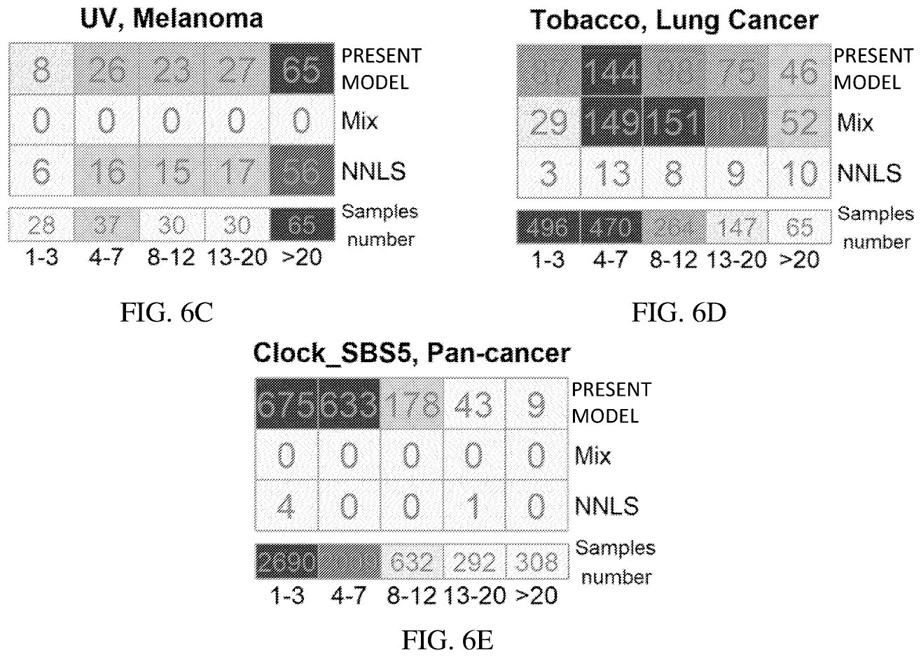

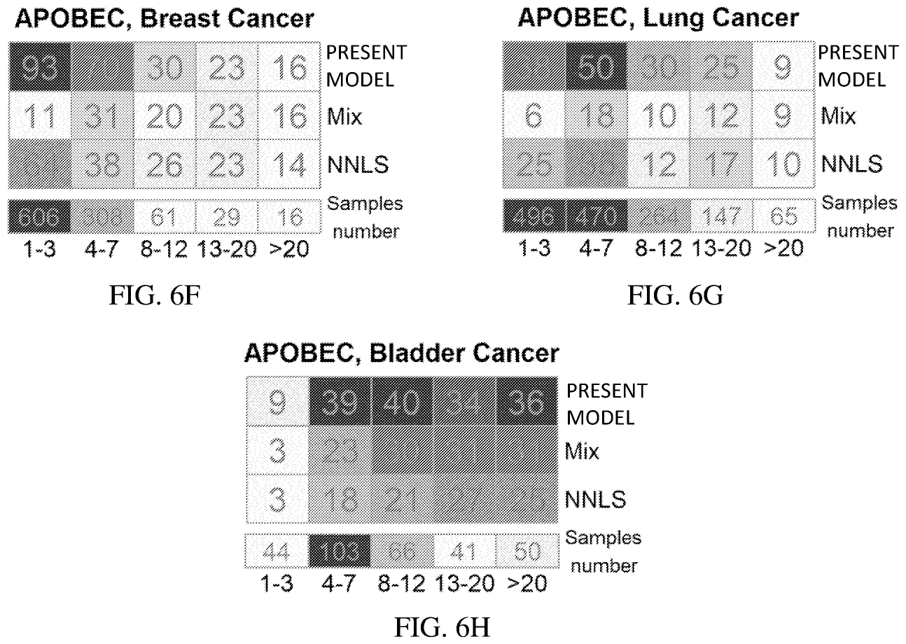

The present inventors used 994 MSK-IMPACT targeted gene panels, annotated with mutational signature indications based on Zehir et al, selecting those samples which had mutation rates (>13.8 mut/Mb) high enough to enable signature analysis. The labels and signature distribution used in this dataset are shown in FIG. 6A.

FIG. 6B shows sensitivity, specificity, PPV and NPV scores of the model's predictions in MSK-IMPACT samples, according to the labels shown in FIG. 6A.

The MSK-IMPACT cohort consists of an additional ~9000 samples without available mutational signature indications. The present inventors inferenced the present model on these samples as follows:
(i) Testing tissue-signature relationships (UV in skin cancer; Tobacco in lung cancer; and APOBEC in breast and bladder cancers),
(ii) comparing these landscapes to the landscape of dominant signatures across WGS cohorts, and
(iii) comparing performance with two common signature fitting methods:
   a. Non-negative least squares (NNLS), and
   b. Mix (see, I. Sason, et al. A mixture model for signature discovery from sparse mutation data. Genome Medicine. 13 (2021), doi:10.1186/s13073-021-00988-7).

The present model was inferenced on all MSK-IMPACT samples having at least 4 mutations. Overall, in each cancer type, the expected signatures indeed had the largest proportions: UV in skin cancers, Tobacco in lung cancers, Clock_SBS5 across many cancers, etc.

Then, as comparison between the results of the present model and NNLS/Mix, the samples were categorized into 5 groups, based on the number of mutations in each sample: 1-3, 4-7, 8-12, 13-20 and more than 20 mutations, the present model predicted a greater number of signatures in the expected cancer type compared to NNLS, and except for the Tobacco signature, also compared to Mix. The results are shown in FIGS. 6C-6H. Importantly, the present model predicted 1,538 samples as Clock_SBS5. This is a ubiquitous, pan-cancer signature. In contrast, Mix and NNLS predicted 0 and 5 samples as Clock_SBS5, respectively.

### Predicting Signature Label From MSK-MET Cohort

The present inventors further used sequencing samples obtained from the MSK-MET cohort to construct an additional validation dataset.

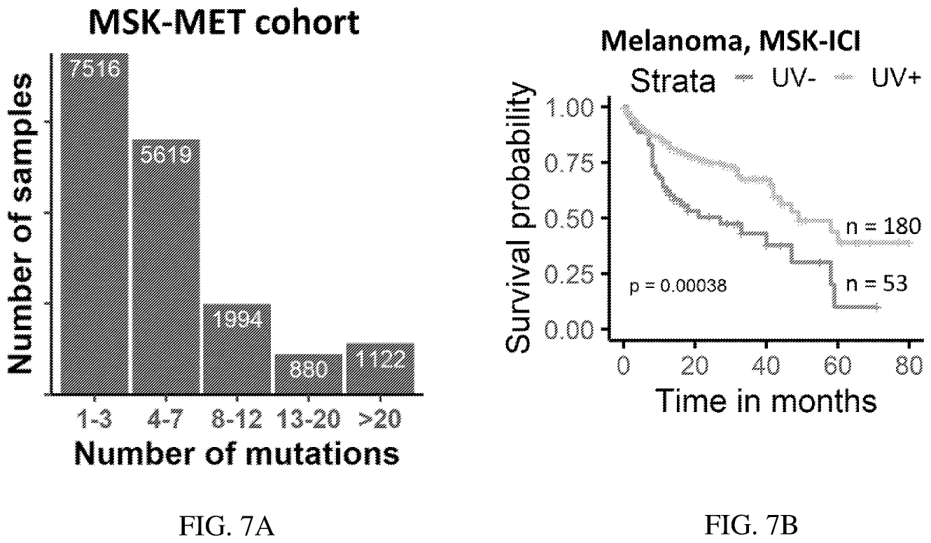

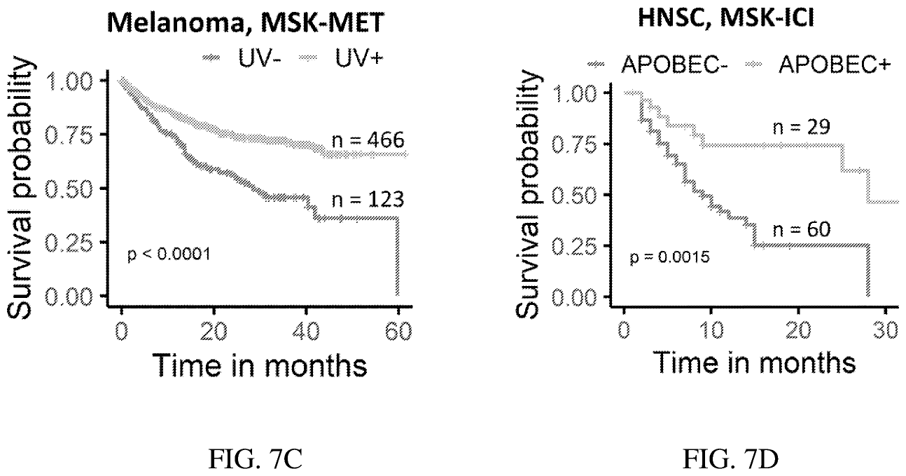

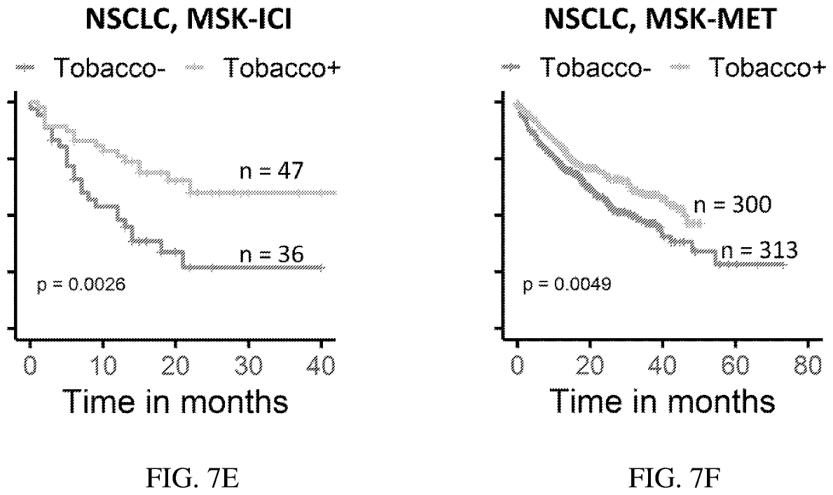

FIG. 7A is a bar plot showing the number of samples from the MSK-MET cohort, respectively, by mutations number.

The MSK-MET dataset comprised 9,615 samples having at least 4 mutations each. The present machine learning model was inferenced on these samples. The distribution of signature predictions across cancer types showed similar results to the MSK-IMPACT cohort and the PCAWG WGS samples reported above. Notably, more than 75% of skin cancer samples had UV signature; Tobacco was most prevalent in lung cancers; APOBEC was most prevalent in bladder, breast, head and neck, and thyroid cancers; MMR was most prevalent in endometrial, colorectal, and prostate samples; and POLE was most prevalent in endometrial and colorectal samples, as expected. Only cancer types with at least 50 samples in MSK-IMPACT were included.

For samples with greater than 3 mutations, the results are highly similar to those of MSK-IMPACT and MSK-MET cohorts reported above. This both strengthens the prediction results and provides a landscape of these signatures across a total of more than 60,000 samples in total. For the 2-3 and single mutation groups, to reduce the false positive rates, the minimum cosine threshold was increased from 60% to 80%, such that a given sample's mutational profiles embeddings need to be 80% or more similar to the pre-learned signature embeddings, to output a prediction. Here, most of the samples were left without prediction. However, the dominant signature in skin cancers from the 2-3 mutations group was still the UV signature, and the dominant signature in bladder, breast, and head and neck cancers was APOBEC. In the single mutation group, while the rates of false positive were significantly higher, Tobacco and APOBEC signatures were still the dominant ones in their expected cancer types.

The present inventors constructed a further validation dataset comprising 1,661 MSK-based samples of patients who received immunotherapy. These samples, named MSK-ICI, are from both MSK-IMPACT and MSK-MET cohorts. Since MSK-ICI patients are a subset of MSK-MET, duplicate samples with the MSK-MET cohort were removed.

The results are shown in FIGS. 7B-7F, which are Kaplan-Meyer plots of overall survival in patients treated with ICI (MSK-ICI), or in the MSK-MET cohort, stratified by a specific signature detected by the present machine learning model. FIG. 7B shows the results of melanoma, UV signature, MSK-ICI cohort. FIG. 7C shows the results for melanoma, UV signature, MSK-MET cohort. FIG. 7D shows the results for head and neck cancer, APOBEC signature, MSK-ICI cohort. FIG. 7E shows the results for NSCLC, Tobacco signature, MSK-ICI cohort. FIG. 7C shows the results for NSCLC, Tobacco signature, MSK-MET cohort.

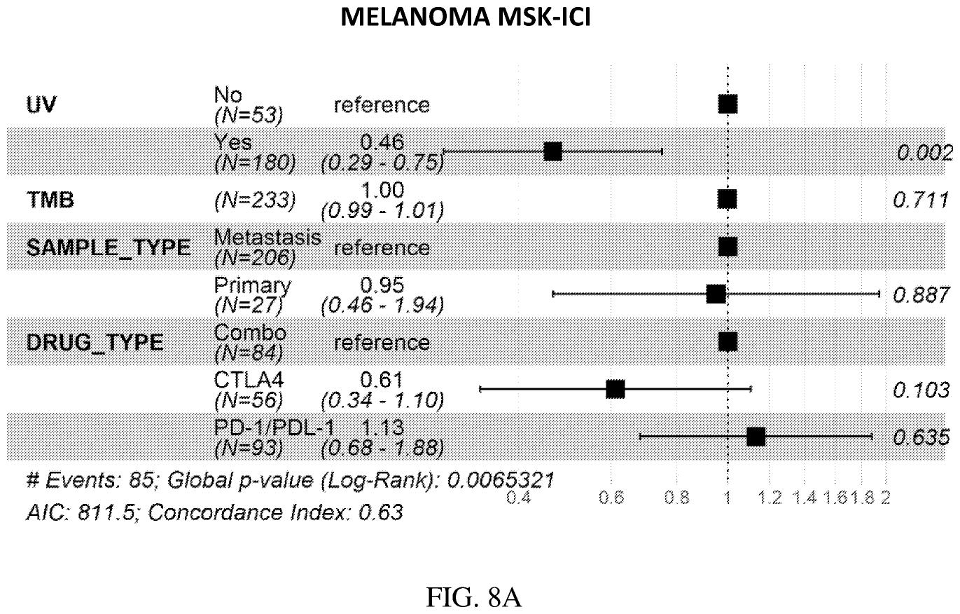

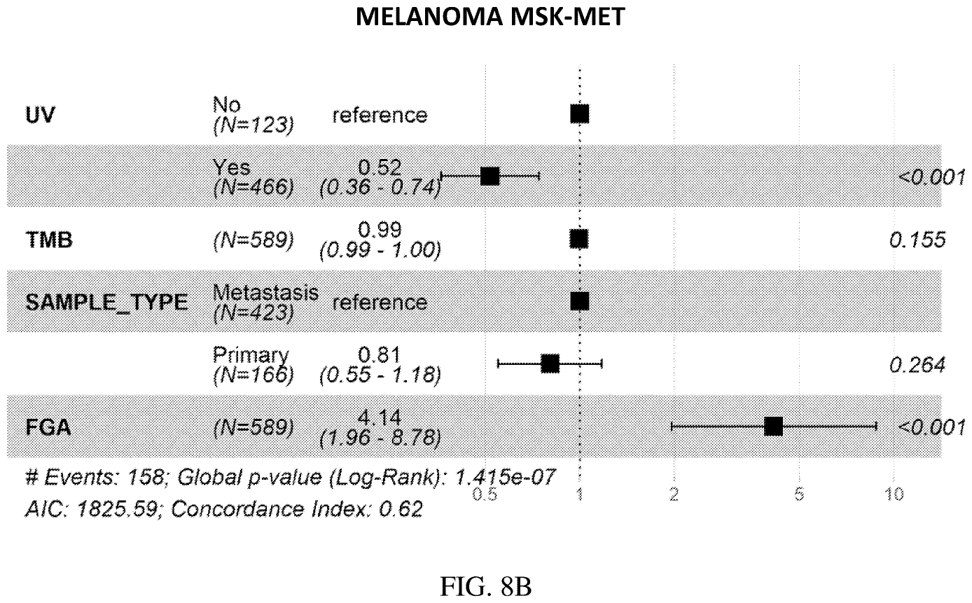

FIGS. 8A-8B show multivariate survival analysis for melanoma, UV signatures, MSK-ICI (FIG. 8A) and MSK-MET (FIG. 8B).

In melanoma, 232 samples had at least 4 mutations. The samples predicted by the present model to be UV-positive had better overall survival rates, both in a univariate (p=0.00038, Cox proportional-hazards) and multivariate (Hazard ratio of 0.46, CI 0.29-0.75, p=0.002), independently from tumor mutational burden (TMB), primary/metastatic status and ICI (immune checkpoint inhibitor) type (FIG. 7B, 8A).

Significantly, in MSK-MET samples which are not part of MSK-ICI, and without treatment information, UV positive samples were again strongly associated with better prognosis, both in univariate (p<0.0001) and multivariate analyses (Hazard ratio of 0.52, CI 0.36-0.74, p<0.001) (FIGS. 7C, 8B). In addition, the present model's UV predictions better differentiate the survival rates than using Zehir et al. annotations.

In MSK-ICI, APOBEC predicted samples in Head and Neck cancer had better survival rates than non-APOBEC samples in a univariate and multivariate analyses (p=0.0015 and p=0.004, respectively) (FIG. 7D).

In MSK-MET there were no significant survival differences for APOBEC samples in head and neck cancer. This might be due to different treatment regimens, different subtypes distribution, or both. Lastly, Tobacco signature positive samples were also associated with better prognosis in NSCLC, both in MSK-ICI and MSK-MET, and both in univariate (p=0.0026 and p=0.0049) and multivariate (p=0.014 and p=0.03) analyses (FIGS. 7E-7F). Due to lower PPV of tobacco relative to UV and APOBEC in NSCLC, only samples with at least 12 mutations were considered, instead of 4 as previously described.

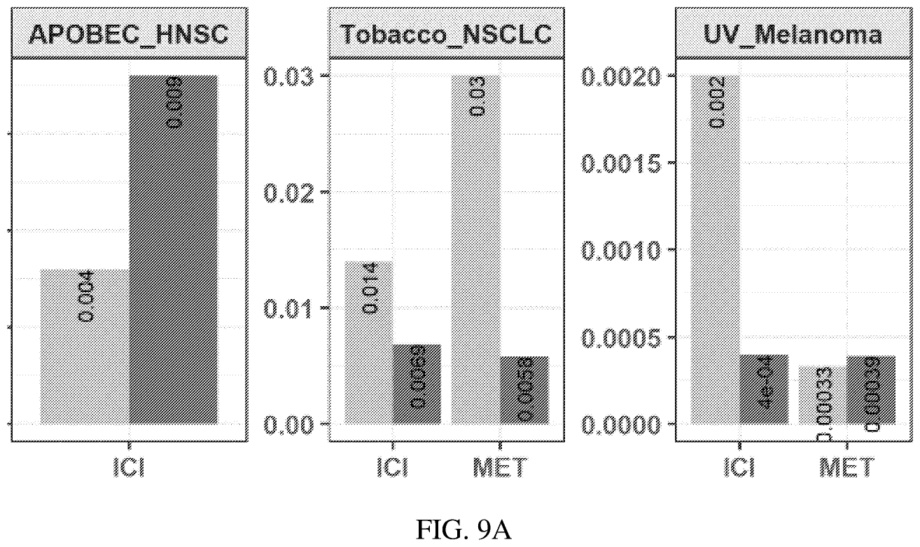

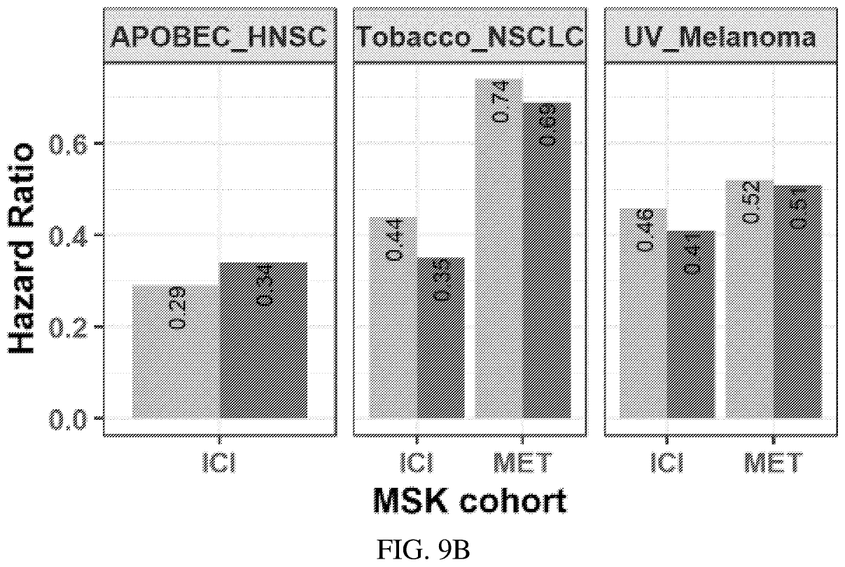

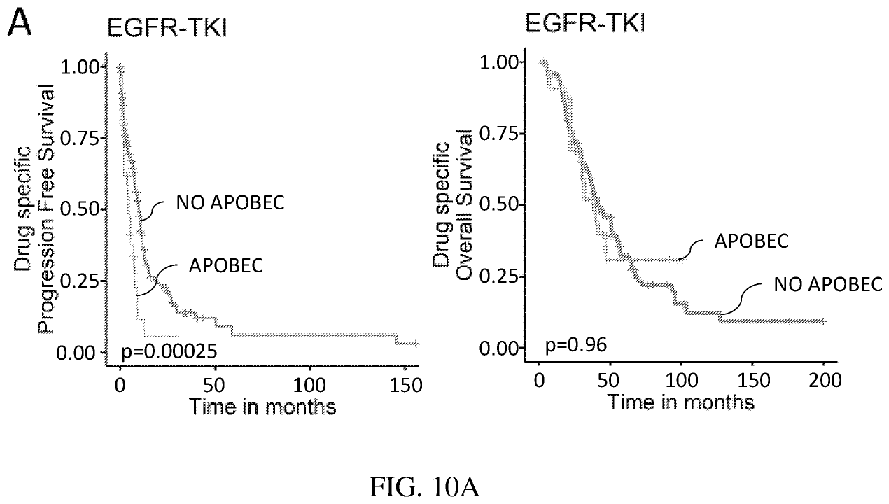

FIGS. 9A-9B present a comparison of p-values (FIG. 9A) and hazard ratio values (FIG. 9B) between results of multivariate survival analysis as presented in FIGS. 7A-7F and FIGS. 8A-8B with the results received by annotating signatures with combinations of two mutational signatures. Except in the case of APOBEC, there are incremental improvements in using the dual-labels approach.

The signature landscape established based on these tests enables analyses to detect associations between specific signatures and cancer genes or hotspot mutations. Many such associations were identified. For example, Clock_SBS5 samples are associated with mutations in TP53, EGFR, KRAS and others. Pan-cancer overall survival analysis based on classification of Clock_SBS5 showed association with poorer prognosis in a pan-cancer manner across independent cohorts and, where available, independent from age. The association was also independent from mutations in TP53 and KRAS, as these genes are considered as negative prognostic markers, and therefore indicates a more general association with survival rather than a mere indicator of age or specific mutated genes. To further explore gene-signature associations, with Clock_SBS5 as an example, a binomial test was carried out to detect positive and negative associations. Different approach is to assess hotspot mutations associations. Such examples are FGFR3^{S249C} with APOBEC in Bladder Cancer (81/107 and 135/224 in MSK-MET and GENIE respectively), and EGFR^{T790M} with Clock_SBS5 in Non-Small Cell Lung Cancer (NSCLC) (24/45 and 121/190) in MSK-MET and GENIE respectively). Positive association was also found with Clock_SBS1. As expected, although Tobacco is a frequent signature in NSCLC, only 1/46 (2.2%) and 2/310 (0.6%) of samples with EGFR^{790M} mutation had Tobacco signature. This was also found for EGFR^{L858R} mutation in NSCLC (all associations P<0.05, exact binomial test, considering the signatures proportion).

### Interpretability of Mutations and Signatures Numerical Representations

One of the challenges in implementing machine learning models in healthcare is the lack of interpretability of many models, which frequently are considered “black boxes.” Interpretability of models not only paves the way to using these models, but gives opportunity to deduce additional insights rather than the prediction itself.

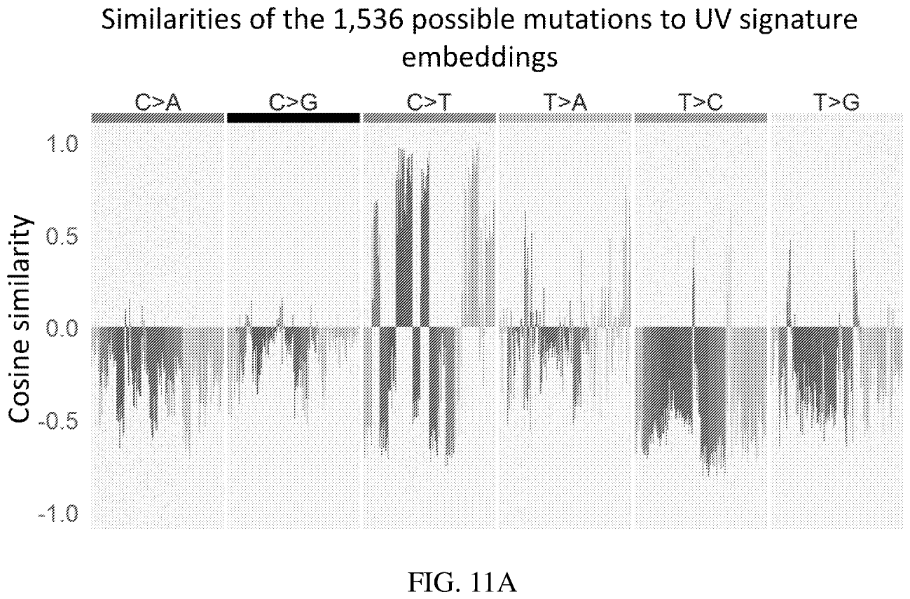

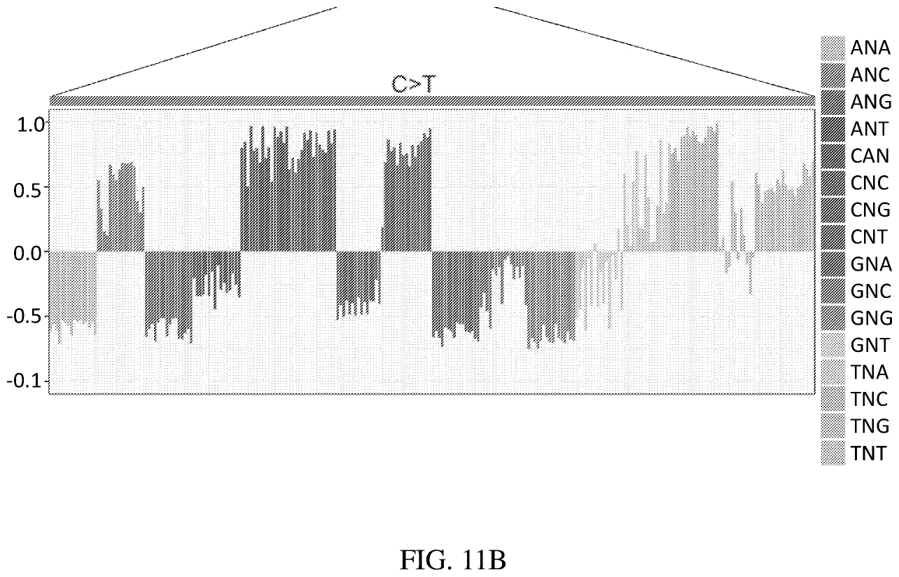

In the present case, it is shown that the pre-learned representations of the present model indeed capture anticipated knowledge, and provided insights of the relations between extended context mutations (1,536 mutations classification) and signatures. Taking UV signature as an example, the present model identified that the cosine similarities between each mutation type, of the 1,536 possible ones, indeed represents what would be expected based on the extensive characterization of UV-related signatures. Briefly, UV damage is characterized mainly by C>T mutations, in trinucleotide contexts of TCA, TCT, TCC, CCC, CCG, and CCT. These mutation classes embedding values indeed have a high cosine similarity with the UV signature (see FIGS. 11A-11B). To a lesser extent, some T>A, T>C and T>G mutations can be introduced by UV damage to the DNA (SBS7c-d) (2). The embeddings of these mutations, such as T>A at TTT in SBS7c, also share similarities with the UV signature embeddings (FIG. 11A). Since penta-nucleotide context was used, for each trinucleotide there are 16 possibilities. Interestingly, not all 16 options get similar embeddings. For example, in one of the landmark mutations of SBS7a, C>T at TCA, only a subset of mutations had high similarity with UV signature, while others are not indicative of the UV embeddings (FIG. 11B). This is also true for T>A at TTT for example (FIG. 11A), and for many other mutations in other signatures such as C>T at TCA in APOBEC, C>A at TCA in POLE. Taken together, these findings not only enable interpretability of the present model results, but provide insights into the penta-nucleotide importance for signatures formation. Interestingly, the higher specificity of each mutation using the 1,536 classification might explain the prediction abilities of the present model with only a few mutations.

### Clinical Use-Case: Non-Small Cell Lung Cancer (NSCLC)

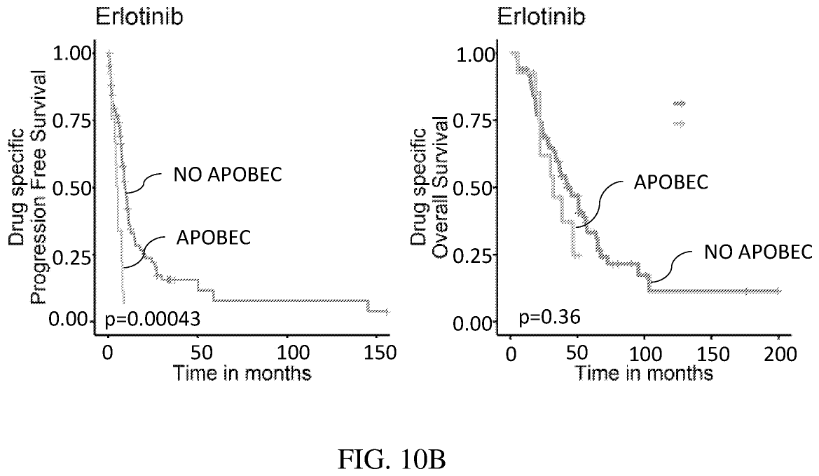

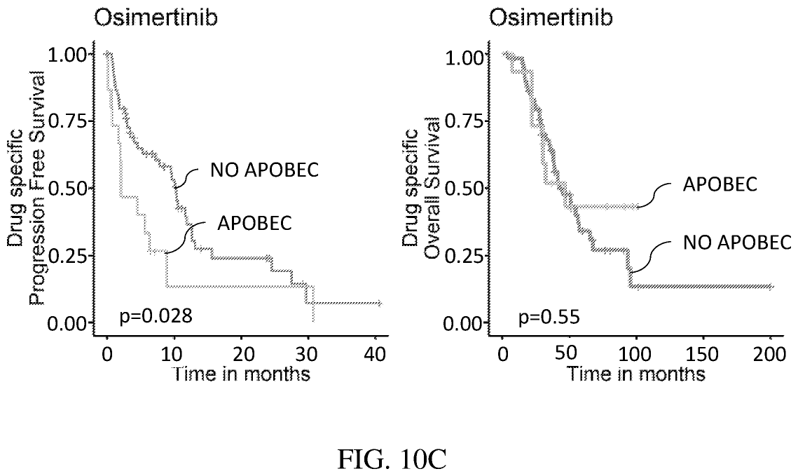

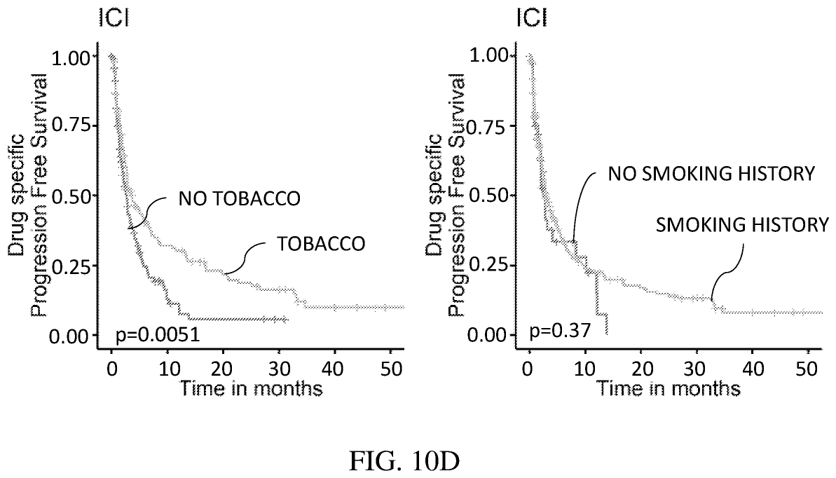

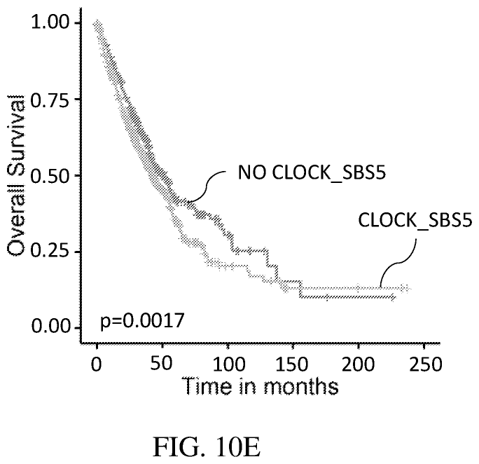

FIGS. 10A-10E show survival rates of NSCLC patients.

FIG. 10A shows stratification of patients treated by any EGFR-TKI whose samples were associated with an APOBEC mutational signature prediction, in drug-specific progression free survival (left panel), and overall survival (right panel). FIG. 10B shows stratification of patients treated by Erlotinib whose samples were associated with an APOBEC mutational signature prediction, in drug-specific progression free survival (left panel) and overall survival (right panel). FIG. 10B shows stratification of patients treated by Osimertinib whose samples were associated with an APOBEC mutational signature prediction, in drug-specific progression free survival (left panel) and overall survival (right panel). FIG. 10D shows drug-specific progression free survival analysis in patients treated by an ICI, whose samples were associated with a TOBACCO mutational signature prediction (left panel) and smoking history (right panel). FIG. 10E shows overall survival analysis at the cohort-level, for patients whose samples were associated with a Clock_SBS5.

The NSCLC GENIE-BPC data contains 1,846 NSCLC patients from four institutions, with available genomic data (different targeted gene panels), detailed treatment history, treatment-specific progression-free survival (PFS), overall survival (OS), and additional elaborative clinical data (see, Lavery, J. A. et al. A Scalable Quality Assurance Process for Curating Oncology Electronic Health Records: The Project GENIE Biopharma Collaborative Approach. JCO Clin Cancer Inform (2022) doi:10.1200/CCI.21.00105).

The present machine learning model was inferenced on this dataset and was able to predict mutational signatures across the GENIE-BPC cohort. In this group, the predictions of the present model were 49 Tobacco, 30 Clock_SBS5, 13 APOBEC, 3 Clock_SBS1, 1 MMR, and no POLE, HRD, or UV.

The present model found that prediction of APOBEC signature is a robust and strong predictive marker for resistance in a patient to EGFR-TKIs, such as Erlotinib and Osimertinib (FIG. 10A-10C). For all EGFR-TKIs combined (i.e., Erlotinib, Osimertinib and Afatinib), the treatment-specific PFS was significantly shorter in APOBEC positive patients than in negative patients (P=0.00025, Cox proportional hazards). This is a predictive biomarker rather than prognostic as the OS in these patients does not depend on APOBEC. Hence, this is an indication of APOBEC-mediated resistance to EGFR-TKIs. This was shown to be true also when examining Erlotinib and Osimertinib separately (PFS: P<0.05; OS: P>0.05), while for Afatinib the patient population is too small (n=27, 4 of them APOBEC positive patients) to be examined by itself (FIGS. 10B-10C). No other group of treatments (i.e., ICI, chemotherapy, chemotherapy+bevacizumab, and ALK/ROS1 inhibitors) showed APOBEC-mediated resistance.

The present model found Tobacco signature to be a positive predictive marker for ICI response, both in terms of treatment-specific PFS and OS, and even when excluding MSK patients which have been included in previous analyses of OS (P<0.05, FIG. 10D, left panel). In contrast, clinical annotation of smoking history is not sufficient to reproduce this association (FIG. 10D, right panel). Hence, the present model provides for a better prediction for Tobacco-associated mutagenesis process than clinical annotation of smoking, and is a robust positive prognostic marker for ICI treatment.

Lastly, from a cohort perspective, Clock_SBS5 prediction was shown to be a prognostic factor, independently from both age and stage (Hazard ratio of 1.4, 1.13-1.6 CI, FIG. 10E). The association is also statistically significant when excluding MSK-based patients (Hazard ratio of 1.3, 1.05-1.7 CI).

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire. Rather, the computer readable storage medium is a non-transient (i.e., not-volatile) medium.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object-oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, a field-programmable gate array (FPGA), or a programmable logic array (PLA) may execute the computer readable program instructions by utilizing state information of the computer readable program instructions to personalize the electronic circuitry, in order to perform aspects of the present invention. In some embodiments, electronic circuitry including, for example, an application-specific integrated circuit (ASIC), may be incorporate the computer readable program instructions already at time of fabrication, such that the ASIC is configured to execute these instructions without programming.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention.

In this regard, each block in the flowchart or block diagrams may represent a module, segment, or portion of instructions, which comprises one or more executable instructions for implementing the specified logical function(s). It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

In the description and claims, each of the terms “substantially,” “essentially,” and forms thereof, when describing a numerical value, means up to a 20% deviation (namely, ±20%) from that value. Similarly, when such a term describes a numerical range, it means up to a 20% broader range −10% over that explicit range and 10% below it).

In the description, any given numerical range should be considered to have specifically disclosed all the possible subranges as well as individual numerical values within that range, such that each such subrange and individual numerical value constitutes an embodiment of the invention. This applies regardless of the breadth of the range. For example, description of a range of integers from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 4, and 6. Similarly, description of a range of fractions, for example from 0.6 to 1.1, should be considered to have specifically disclosed subranges such as from 0.6 to 0.9, from 0.7 to 1.1, from 0.9 to 1, from 0.8 to 0.9, from 0.6 to 1.1, from 1 to 1.1 etc., as well as individual numbers within that range, for example 0.7, 1, and 1.1.

The descriptions of the various embodiments of the present invention have been presented for purposes of illustration, but are not intended to be exhaustive or limited to the explicit descriptions. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

In the description and claims of the application, each of the words “comprise,” “include,” and “have,” as well as forms thereof, are not necessarily limited to members in a list with which the words may be associated.

Where there are inconsistencies between the description and any document incorporated by reference or otherwise relied upon, it is intended that the present description controls.

1. A computer-implemented method comprising:
receiving, as input, a plurality of DNA sequencing samples corresponding to a cohort of subjects having various tumor types;
annotating each of said DNA sequencing samples with annotations indicating (a) one or more mutations represented in said DNA sequencing sample, and (b) at least one dominant mutational signature associated with said DNA sequencing sample;
training a machine learning model on a training dataset comprising: (i) said DNA sequencing samples, and (ii) said annotations, to learn, for each one of said dominant mutational signatures represented in said DNA sequencing samples, a mutational signature embedding within an n-dimensional vector space, wherein said mutational signature embeddings represent contextual relationships between said dominant mutational signatures and said mutations, such that said mutations related to a particular one of said dominant mutational signatures are represented close together within said n-dimensional vector space;
applying said trained machine learning model to a target DNA sequencing sample from a target subject, to generate target embeddings within said n-dimensional vector space, wherein said target embeddings represent mutations in said target DNA sequencing sample; and
predicting a dominant mutational signature associated with said target DNA sequencing sample, based, at least in part, on a similarity between said target embeddings and one or more of said learned mutational signature embeddings.

2. The computer-implemented method of claim 1, wherein said training dataset comprises a plurality of positive pairs, each comprising (i) one of said mutations represented in a said DNA sequencing sample and (ii) said associated dominant mutational signature; and a plurality of randomly-generated negative pairs, each comprising (iii) one of said mutations represented in said DNA sequencing samples and (iv) an unrelated said dominant mutational signature, and wherein said optimizing is based on a loss function which decreases while a positive score associated with said plurality of positive pairs increases, and a negative score associated with said plurality of negative pairs decreases.

3. The computer-implemented method of claim 1, wherein said similarity is based on calculating a similarity score between (i) a mean target embedding of all of said target embeddings, and (ii) each of said mutational signature embeddings.

4. The computer-implemented method of claim 3, wherein said similarity score is based on a cosine similarity calculation.

5. (canceled)

6. The computer-implemented method of claim 1, wherein said annotations further indicate, for at least some of said DNA sequencing samples, at least one of the following categories of annotations: tumor type, tumor site, metastatic site, structural variants, smoking history of said corresponding subject, vital status of said corresponding subject, survival status of said corresponding subject, sex of said corresponding subject, or age of said corresponding subject.

7. The computer-implemented method of claim 1, wherein said annotations with respect to said mutations represented in said DNA sequencing samples are indicated by their 5-mer sequence.

8. The computer-implemented method of claim 1, wherein, with respect to at least some of said DNA sequencing samples, said annotations indicate a combination of two dominant mutational signatures, and wherein said training comprises learning joint signature embeddings for said combinations of two dominant mutational signatures.

9. (canceled)

10. (canceled)

11. A system comprising:
at least one hardware processor; and
a non-transitory computer-readable storage medium having stored thereon program instructions, the program instructions executable by the at least one hardware processor to:
receive, as input, a plurality of DNA sequencing samples corresponding to a cohort of subjects having various tumor types,
annotate each of said DNA sequencing samples with annotations indicating (a) one or more mutations represented in said DNA sequencing sample, and (b) at least one dominant mutational signature associated with said DNA sequencing sample,
train a machine learning model on a training dataset comprising (i) said DNA sequencing samples, and (ii) said annotations, to learn, for each one of said dominant mutational signatures represented in said DNA sequencing samples, a mutational signature embedding within an n-dimensional vector space, wherein said mutational signature embeddings represent contextual relationships between said dominant mutational signatures and said mutations, such that said mutations related to a particular one of said dominant mutational signatures are represented close together within said n-dimensional vector space,
apply said trained machine learning model to a target DNA sequencing sample from a target subject, to generate target embeddings within said n-dimensional vector space, wherein said target embeddings represent mutations in said target DNA sequencing sample; and
predict a dominant mutational signature associated with said target DNA sequencing sample, based, at least in part, on a similarity between said target embeddings and one or more of said learned mutational signature embeddings.

12. The system of claim 11, wherein said training dataset comprises a plurality of positive pairs, each comprising (i) one of said mutations represented in a said DNA sequencing sample and (ii) said associated dominant mutational signature; and a plurality of randomly-generated negative pairs, each comprising (iii) one of said mutations represented in said DNA sequencing samples and (iv) an unrelated said dominant mutational signature, and wherein said optimizing is based on a loss function which decreases while a positive score associated with said plurality of positive pairs increases, and a negative score associated with said plurality of negative pairs decreases.

13. The system of claim 11, wherein said similarity is based on calculating a similarity score between (i) a mean target embedding of all of said target embeddings, and (ii) each of said mutational signature embeddings.

14. The system of claim 13, wherein said similarity score is based on a cosine similarity calculation.

15. (canceled)

16. The system of claim 11, wherein said annotations further indicate, for at least some of said DNA sequencing samples, at least one of the following categories of annotations: tumor type, tumor site, metastatic site, structural variants, smoking history of said corresponding subject, vital status of said corresponding subject, survival status of said corresponding subject, sex of said corresponding subject, or age of said corresponding subject.

17. The system of claim 11, wherein said annotations with respect to said mutations represented in said DNA sequencing samples are indicated by their 5-mer sequence.

18. The system of claim 11, wherein, with respect to at least some of said DNA sequencing samples, said annotations indicate a combination of two dominant mutational signatures, and wherein said training comprises learning joint signature embeddings for said combinations of two dominant mutational signatures.

19. (canceled)

20. (canceled)

21. A computer program product comprising a non-transitory computer-readable storage medium having program instructions embodied therewith, the program instructions executable by at least one hardware processor to:
receive, as input, a plurality of DNA sequencing samples corresponding to a cohort of subjects having various tumor types;
annotate each of said DNA sequencing samples with annotations indicating (a) one or more mutations represented in said DNA sequencing sample, and (b) at least one dominant mutational signature associated with said DNA sequencing sample;
at a training stage, train a machine learning model on a training dataset comprising (i) said DNA sequencing samples, and (ii) said annotations, to learn, for each one of said dominant mutational signatures represented in said DNA sequencing samples, a mutational signature embedding within an n-dimensional vector space, wherein said mutational signature embeddings represent contextual relationships between said dominant mutational signatures and said mutations, such that said mutations related to a particular one of said dominant mutational signatures are represented close together within said n-dimensional vector space;
apply said trained machine learning model to a target DNA sequencing sample from a target subject, to generate target embeddings within said n-dimensional vector space, wherein said target embeddings represent mutations in said target DNA sequencing sample; and
predict a dominant mutational signature associated with said target DNA sequencing sample, based, at least in part, on a similarity between said target embeddings and one or more of said learned mutational signature embeddings.

22. The computer program product of claim 21, wherein said training dataset comprises a plurality of positive pairs, each comprising (i) one of said mutations represented in a said DNA sequencing sample and (ii) said associated dominant mutational signature; and a plurality of randomly-generated negative pairs, each comprising (iii) one of said mutations represented in said DNA sequencing samples and (iv) an unrelated said dominant mutational signature, and wherein said optimizing is based on a loss function which decreases while a positive score associated with said plurality of positive pairs increases, and a negative score associated with said plurality of negative pairs decreases.

23. The computer program product of claim 21, wherein said similarity is based on calculating a similarity score between (i) a mean target embedding of all of said target embeddings, and (ii) each of said mutational signature embeddings.

24. (canceled)

25. (canceled)

26. The computer program product of claim 21, wherein said annotations further indicate, for at least some of said DNA sequencing samples, at least one of the following categories of annotations: tumor type, tumor site, metastatic site, structural variants, smoking history of said corresponding subject, vital status of said corresponding subject, survival status of said corresponding subject, sex of said corresponding subject, or age of said corresponding subject.

27. The computer program product of claim 21, wherein said annotations with respect to said mutations represented in said DNA sequencing samples are indicated by their 5-mer sequence.

28. The computer program product of claim 21, wherein, with respect to at least some of said DNA sequencing samples, said annotations indicate a combination of two dominant mutational signatures, and wherein said training comprises learning joint signature embeddings for said combinations of two dominant mutational signatures.

29. (canceled)

30. (canceled)

31. The computer-implemented method of claim 1, further comprising performing at least one of the following steps:
administering a cancer treatment to said target subject, based, at least in part, on said predicted dominant mutational signature,
predicting survival in said target subject, based, at least in part, on said predicted dominant mutational signature, or
predicting immunotherapy response in said target subject, based, at least in part, on said predicted dominant mutational signature.

32.-36. (canceled)
